(19) United States
(12) Patent Application Publication
(10) Pub. No.: US 2009/0196929 A1
(43) Pub. Date: Aug. 6, 2009

(54) Silica Wetcake Treatment Method

(75) Inventors: Duen-Wu Hua, Edgewood, MD (US); Michael William Mullahey, JR., Nottingham, MD (US)

Correspondence Address: WYATT, TARRANT & COMBS, LLP, 1715 AARON BRENNER DRIVE, SUITE 800, MEMPHIS, TN, 38120-4367, US

(73) Assignee: J.M. HUBER CORPORATION, Edison, NJ (US)

(21) Appl. No.: 12/024,154

(22) Filed: Feb. 1, 2008

(51) Int. Cl. A61K 9/14; A61K 8/02; C01B 33/14; A61Q 11/00

(52) U.S. Cl. 424/489; 424/49; 423/335

(57) ABSTRACT

New methods of treating silica wetcake during precipitated silica materials manufacturing are provided. Such methods permit a significant increase in high solids content processing while simultaneously reducing high viscosity of the resultant particles for transport facilitation. The resultant precipitated silica wetcake is treated with a borate-containing dispersant to impart the necessary low viscosity characteristics thereto. Such a dispersant accords not only such a viscosity result, but will not char or otherwise discolor the silica particles during evaporation of the liquids within the wetcake itself. Furthermore, such a dispersant, if left on the surfaces of such particles, will not deleteriously affect the abrasivity, fluoride compatibility, or other dentifrice properties of the precipitated silica materials themselves. Also encompassed within this invention are the resultant precipitated silica particles exhibiting borate residues and dentifrices including such materials.

### FIELD OF THE INVENTION

New methods of treating silica wetcake during precipitated silica materials manufacturing are provided. Such methods permit a significant increase in high solids content processing while simultaneously reducing high viscosity of the resultant particles for transport facilitation. The resultant precipitated silica wetcake is treated with a borate-containing or polyphosphate dispersant to impart the necessary low viscosity characteristics thereto. Such a dispersant accords not only such a viscosity result, but will not char or otherwise discolor the silica particles during evaporation of the liquids within the wetcake itself. Furthermore, such a dispersant, if left on the surfaces of such particles, will not deleteriously affect the abrasivity, fluoride compatibility, or other dentifrice properties of the precipitated silica materials themselves. Also encompassed within this invention are the resultant precipitated silica particles exhibiting borate residues and dentifrices including such materials.

### BACKGROUND OF THE INVENTION

An abrasive substance has been included in conventional dentifrice compositions in order to remove various deposits, including pellicle film, from the surface of teeth. Pellicle film is tightly adherent and often contains brown or yellow pigments which impart an unsightly appearance to the teeth. While cleaning is important, the abrasive should not be so aggressive so as to damage the teeth. Ideally, an effective dentifrice abrasive material maximizes pellicle film removal while causing minimal abrasion and damage to the hard tooth tissues. Consequently, among other things, the performance of the dentifrice is highly sensitive to the extent of abrasion caused by the abrasive ingredient. Conventionally, the abrasive cleaning material has been introduced in flowable dry powder form to dentifrice compositions, or via redispersions of flowable dry powder forms of the polishing agent prepared before or at the time of formulating the dentifrice.

Synthetic low-structure precipitated silicas have been utilized for such a purpose due to the effectiveness such materials provide as abrasives, as well as low toxicity characteristics and compatibility with other dentifrice components, such as sodium fluoride, as one example. When preparing synthetic precipitated silicas, the objective is to obtain silicas which provide maximal cleaning with minimal impact to the hard tooth surfaces.

Generally, dentifrices comprise a majority of a humectant, such as sorbitol, glycerin, polyethylene glycol, and the like, in order to permit proper contact with target dental subjects, an abrasive such as precipitated silica for proper cleaning and abrading of the subject teeth, water, and other active components such as fluoride-based compounds for anticaries benefits. The ability to impart proper rheological benefits to such a dentifrice is accorded through the proper selection and utilization of thickening agents, such as hydrated silicas, hydrocolloids, gums, and the like, to form a proper network of support to properly contain such important humectant, abrasive, and anticaries ingredients. It is thus evident that formulating proper dentifrice compositions can be rather complex, both from a compounding standpoint as well as the number, amount, and type of components present within such Formulations. As a result, the ability to provide an effective abrasive that is compatible with such dentifrice components and not exhibit deleterious effects therein such Formulations, is of great necessity.

Synthetically produced precipitated low-structure silicas, in particular, have been used as abrasive components in dentifrice Formulations due to their cleaning ability, relative safeness, and compatibility with typical dentifrice ingredients, such as humectants, thickening agents, flavoring agents, anticaries agents, and so forth. As known, synthetic precipitated silicas generally are produced by the destabilization and precipitation of amorphous silica from soluble alkaline silicate by the addition of a mineral acid and/or acid gases under conditions in which primary particles initially formed tend to associate with each other to form a plurality of aggregates (i.e., discrete clusters of primary particles), but without agglomeration into a three-dimensional gel structure. The resulting precipitate is separated from the aqueous fraction of the reaction mixture by filtering, washing, and drying procedures, and then the dried product is mechanically comminuted in order to provide a suitable particle size and size distribution. The silica drying procedures are conventionally accomplished using spray drying, nozzle drying (e.g., tower or fountain), wheel drying, flash drying, rotary wheel drying, oven/fluid bed drying, and the like. Additionally, precipitated silicas intended for dentifrices require comminution in order to reduce the particle size of the dried precipitated silica product down to a size that does not feel gritty in the mouth of a dentifrice user, while, on the other hand, not being so small as to lack sufficient polishing or thickening action. That is, in conventional practice, the median particle size of the silica in the reactor formed by acidulation of a metal silicate is too large for dentifrice applications and the like. To comminute silica particulates, grinding and milling equipment are used, such as a hammer or a pendulum mill used in one or multiple passes, and fine grinding has been performed, for example, by fluid energy or air-jet mill.

One way of reducing the costs involved with dentifrice manufacture is to provide a high solids-content precipitated silica wetcake that does not require substantial drying times and temperatures. As the costs associated with such necessary evaporation processes has proven to be rather expensive (and only increases as energy production costs themselves increase), the lower the amount of water (or other liquid) present within the precipitated silica wetcake, the lower the time and/or temperature required to dry the particles present therein. Thus, there is a desire to reduce the amount of water (or other liquid) within the produced silica wetcake during abrasive silica production methods.

Furthermore, this drive to higher solids content wetcake materials is also tempered with the need to provide an effective means for transporting such high solids materials prior to evaporation. It is thus necessary to treat the silica solid particles in some manner to reduce the viscosity of the solids to the extent that such can be moved easily (such as via a pump through a pipe or into a rail car or like transport device) to a drying device and delivered to the dentifrice manufacturer for incorporation with such an intended end-use composition.

This reduction in viscosity has been accomplished in the past for different non-abrasive precipitated silicas through the inclusion of certain dispersant materials, such as aluminum compounds and aluminum salts. However, these dispersant types have proven ineffective for silica abrasives due to fluoride compatibility problems and other potential compatibility issues with dentifrice ingredients. Other types of dispersants have been attempted for abrasive silicas (such as humectants and sorbitol-like compounds). Unfortunately, these compounds, that do tend to provide effective viscosity modification and acceptable dentifrice compatibility, exhibit scorching and charring upon exposure to drying temperatures. As such, since such dispersants must be introduced prior to the drying step for effectiveness, such alternative methods have been avoided due to discoloration results.

Therefore, a manufacturing method for precipitated silica materials that can provide effective low viscosity to silica particles during the wetcake production step in such a manner that dentifrice color and ingredient compatibility is not compromised is a highly desired result. To date, however, no such method has been provided the precipitated silica abrasive materials industry.

### BRIEF DESCRIPTION AND ADVANTAGES OF THE INVENTION

It has now been realized that the inclusion of a borate compound during precipitated silica wetcake treatment provides an effective manner of imparting a high viscosity dispersant to such materials that will not degrade or char during drying and exhibits no appreciable incompatibility with dentifrice ingredients. Accordingly, this invention thus encompasses such a wetcake treatment method as well as precipitated silica particles that exhibit abrasivity and include some borate content thereon.

The advantages of such a method (as well as the advantages exhibited by such borate- and/or polyphosphate-treated treated particles) include the ability to provide a high solids-content wetcake, the particles of which are easily transferred due to low viscosity imparted by the borate. Furthermore, another advantage of this method and resultant particles is the compatibility of borate- and/or polyphosphate-treated silica with dentifrice components, such as fluorides, viscosity modifiers, humectants, and the like, as well as the ability of such borates and/or polyphosphates to withstand temperatures generally utilized to further evaporate the moisture content of such high solids-content precipitated silica particles.

Overall, the invention encompasses a method for preparing precipitated silica particulate materials, said method comprising the steps of:

a) producing a wetcake of precipitated silica;

b) treating said wetcake of step “a” with from 0.001 to 3% by weight thereof said wetcake of an aqueous solution of a borate-containing and/or polyphosphate compound; and

c) mixing said treated wetcake into a homogeneous slurry of treated particles of borate-treated and/or polyphosphate-treated precipitated silica.

This method may further include the steps of:

d) drying said treated particles of step “c” to a solids content of at most 99%;

e) comminuting said dried individual particles; and

f) incorporating either said treated particles of step “c” or said comminuted individual particles of step “e” into a dentifrice composition.

Also encompassed within this invention is thus a dentifrice including such borate-treated and/or polyphosphate-treated precipitated silica particles as well as the borate-treated and/or polyphosphate-treated precipitated silica particles themselves.

Preferably, the comminuting used in the above-mentioned various embodiments of the invention is accomplished by grinding and milling equipment, such as a hammer or a pendulum mill, and fine grinding by, for example a fluid energy or air-jet mill, either as a single stage or multi-stage procedure.

The method of the invention can be practiced more economically because the ability to transfer high solids-content particles permits quicker drying times ultimately, as the necessity of evaporating liquid from the target particles is reduced with an initial high solids content material.

### DETAILED DESCRIPTION OF THE PREFERRED EMBODIMENTS

All parts, percentages and ratios used herein are expressed by weight unless otherwise specified. All documents cited herein are incorporated by reference. The following describes preferred embodiments of the present invention, which provides silica for use as abrasive or thickening agents in dentifrices, such as toothpastes, or silicates as active or added ingredients within various types of compositions as well. While the optimal use for this silica is in dentifrices, this silica may also be used in a variety of other consumer products.

By “mixture” it is meant any combination of two or more substances, in the form of, for example without intending to be limiting, a heterogeneous mixture, a suspension, a solution, a sol, a gel, a dispersion, or an emulsion.

By “dentifrices” it is meant oral care products such as, without intending to be limiting, toothpastes, tooth powders and denture creams.

A generalized processing scheme for precipitated silica production is as follows:

In the first step of the processing scheme, an acidulation reaction is performed to precipitate silica. The initial acidulation reaction is performed in a reaction system equipped with suitable heating equipment. In general, the produced precipitated silicas may be prepared by a fresh water, or electrolyte solution, acidulation process wherein silica is precipitated by reaction of an alkali metal silicate and a mineral acid in aqueous solution. In the fresh water process, no electrolyte such as alum, Na₂SO₄, or NaCl, is present during the acidulation reaction.

Sodium silicate solution is charged to a reactor container or chamber including agitator to serve as initiating nuclei for the silica. The aqueous solution of sodium silicate in the container is then preheated to a temperature in the range of about 60 to 100° C., more preferably about 70 to 95° C. Prior to introduction into the reactor container, any remaining sodium silicate that may be added is preferably preheated to about 70 to 95° C. An acid solution is preferably preheated to about 30 to 35° C.

Although sodium silicate is illustrated, it will be understood that any suitable alkali metal silicate could be used. The term “alkali metal silicate” includes all the conventional forms of alkali silicates, as for example, metal silicates, disilicates and the like. Water soluble potassium silicates and sodium silicates are particularly advantageous with the latter being preferred. It should be taken into consideration that the mole ratio of the alkali silicate, i.e., the ratio of silica to alkali metal oxide, contributes, depending on other reaction parameters, to the average pore size of the silica products. In general, acceptable silica products of this invention can be made with silicate molar ratios (SiO₂:Na₂O) ranging from about 1.0 to 3.5 and preferably from about 2.4 to about 3.4. The alkali silicate solution supplied to the reactor vessel during various processing steps in the inventive method, as described elsewhere herein, generally can contain between about 8 to 35%, and more preferably between about 8.0% and 15.0%, by weight alkali metal silicate based on the total weight of the alkali metal silicate solution. In order to reduce the alkali silicate concentration of a source solution of alkali silicate to the above-indicated desired range, dilution water can be added to a source solution of alkali silicate before the silicate solution is fed into the reactor, or, alternatively, the dilution water can be combined in situ with the source solution of alkali silicate in the reactor used in the acidulation reaction step with agitation-mixing to formulate the desired concentration of silicate in the alkali metal silicate solution.

The acid, or acidulating agent, can be a Lewis acid or Brönsted acid, and preferably is a strong mineral acid such as sulfuric acid, hydrochloric acid, nitric acid, phosphoric acid, and so forth, and more preferably sulfuric acid, added as a dilute solution thereof (e.g., at a concentration of between about 6 to 35 wt %, more typically about 9.0 to 15.0 wt %).

Once the reactor solution and remaining reactants have reached the desired temperatures, addition of acid or simultaneous addition of additional sodium silicate solution and acid into the reactor is commenced. The acid and sodium silicate solutions are metered into the reactor over an addition time of about 30 to 90 minutes. Rates of addition of the reactants depend upon the mole ratio, addition time and concentration of the silicate and the concentration of the acid. Generally, 2 moles sodium is neutralized with one mole sulfuric acid.

Optionally a water soluble metal salt adduct material such as a water soluble salt of aluminum, calcium, magnesium or zinc may be mixed with the acidulating agent and introduced into the reaction mixture along with the acidulating agent.

At the end of this addition period, most of the silica has precipitated. Additional acid is metered into the reactor until the reactor slurry reaches the desired pH. Once the slurry pH reaches about 7.0, it is preferable to reduce the acid flow rate until the slurry pH approaches the target pH, at which point the acid flow can be stopped and manual adjustment used to reach the target slurry pH. The preferred slurry pH is approximately 4.0 to 7.0, and more preferably between 4.5 to 6.0. At this juncture, the silica has precipitated to provide a mixture of the precipitated silica and the reaction liquor. Once the desired slurry pH is reached, digestion begins and the reaction temperature is raised to approximately 85-99° C., and preferably 91 to 97° C., and digestion is continued at the elevated temperature for approximately 5 to 60 minutes, and preferably for approximately 10 to 30 minutes. Acid is added during the digestion step to the extent necessary to maintain a constant pH.

After the digestion step is completed in the reactor, and any subsequent pH adjustment conducted, the reaction batch is discharged from the reactor. Although the above-described general protocol are preferred for synthesizing the precipitated silica to be conditioned according to this invention, it will be appreciated that other grades of precipitated silicas, such as very low to very high structure synthetic silicas in accordance with the definitions set forth in J. Soc. Cosmet. Chem., 29, 497-521 (August 1978), and Pigment Handbook: Volume 1, Properties and Economics, 2nd ed., John Wiley & Sons, 1988, p. 139-159, generally can be used in the practice of this invention.

The resultant silica reaction slurry then requires further processing, such as filtering and/or drying to a high solids-content wetcake material. Such a step can be performed in any standard method, including rotary drum vacuum filter, centrifuge filter, filter press, and the like. Once the wetcake is formed, however, the inventive treatment with a borate compound is then performed. The term “a borate compound” is intended to encompass any aqueous solution of an inorganic salt including at least one boron and at least one oxygen. Borax (sodium tetraborate decahydrate) is the most preferred material for this method step. Other potential borate-containing compounds include sodium metaborate and are potentially preferred as well. Furthermore, polyphosphates may be utilized as an alternative or in addition to the preferred borate compounds. These specific types of inorganic dispersants impart the necessarily low viscosity properties to the target silica particles to facilitate transfer of such solid particles to a drying device. Such dry particles, or, if desired, the high solids-content particles, may be further comminuted to any desired particle size. The borate and/or polyphosphate dispersants are added to the wetcake in dry (or in solution with another suitable solvent) form in an amount of from about 0.01 to 3.0% of the total weight of the wetcake.

The treated wetcake is then homogeneously mixed into a slurry that can be more easily transported via pumping or like manner to a drying device for further treatment via moisture reduction, milling, and ultimate dentifrice formulation introduction. Alternatively, the slurry may be introduced directly into a dentifrice formulation as the borate and/or polyphosphate treatment provides improved dispersability and other like properties of the precipitated silica abrasive or thickener materials, as well as reduced viscosity for the target dentifrice if desired.

During dentifrice formulation, the abrasive/thickener precipitated silica particles may be added in any conventional manner. Generally, metered dispensing into a pre-mix formulation is performed for such a purpose. An exemplary toothpaste Formulation is provided below.

As discussed above, the inorganic borate and/or polyphosphate dispersants do not impart a deleterious property to the particulate surfaces in terms of drying temperature exposure or dentifrice component compatibility.

The target mean particle size for the borate-treated and/or polyphosphate-treated precipitated silica particles is between about 1 to about 30 microns (μm), preferably between about 3 and about 17 microns, and yield a free flowing silica powder with less than 12% moisture, preferably less than 9% moisture. The abrasive particles in the milled abrasive composition have less than 2.0 wt % fraction of +325 mesh size particles (greater than about 45 μm) as well.

In addition to the above-described methodology of precipitating the raw synthetic amorphous silicas in the reactor, the preparation of the raw silica is not necessarily limited thereto and it also can be generally accomplished in accordance with the methodologies described, for example, in prior U.S. Pat. Nos. 3,893,840, 3,988,162, 4,038,098, 4,067,746, 4,340,583, 4,420,312, 5,225,177 and 5,891,421, all of which are incorporated herein by reference. The precipitated silica compositions of this invention generally have the following properties: linseed oil absorptions between about 40 to about 230 cc/100 g, RDA (Radioactive Dentin Abrasion) values between about 20 to about 250, and a % Transmittance (% T) greater than about 20.

Examples of potential dentifrice end-uses are described herein and/or, for example, in Reissue 29,634, and U.S. Pat. Nos. 5,676,932, 6,074,629, and 5,658,553, and the patents cited therein, all being incorporated herein by reference. For such dentifrice purposes, the inventive borate- and/or polyphosphate-treated precipitated silica particles, when incorporated into dentifrice compositions such as toothpaste, are present at a level of from about 1 to about 50% by weight, more preferably from about 1 to about 35% by weight in the Formulation.

The presence of other silica materials, in addition to the type produced via the inventive method described herein, as well as two or more silica abrasives and/or silica thickeners made via such a method, are encompassed within the term “abrasives” within the possible dentifrice formulations encompassed within this invention. The term “Therapeutic agents” includes materials such as, without limitation, antimicrobial agents (cationic, anionic and nonionic) and anticaries agents as well as any other type of typical component within dentifrice Formulations that provide therapeutic effects to the teeth and/or gums of the user. Suitable antimicrobial agents include bisguanides, such as alexidine, chlorhexidine and chlorhexidine gluconate; quarternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sodium monofluorophosphate, stannous fluoride, and the like sanguinaria extract and sanguinarine; volatile oils, such as eucalyptol, menthol, thymol, and methyl salicylate; amine fluorides; peroxides and the like. Therapeutic agents may be used in dentifrice formulations singly or in combination.

The following examples are presented to illustrate the invention, but the invention is not to be considered as limited thereto. In the following examples, parts are by weight unless indicated otherwise.

### Precipitated Silica Production

### INITIAL EXAMPLE I

46.6 liters of 13.3% 2.65 MR sodium silicate were introduced within a reactor and agitated at 80 rpm while heated to 95° C. with a recirculation rate of 75 liters per minute begun after 5 minutes addition time passed. At that time, simultaneous addition of sodium silicate (13.3%, 85° C.) and sulfuric acid (11.4%) was initiated with the silicate introduced at a rate of 11.41 l/min and the acid at 5.35 l/min. After 6 minutes of addition time, the silicate addition and acid flow were terminated. 58 liters of water were then added to the reactor and the reactor was reheated to 95° C. Subsequently, the silicate and acid simultaneous addition was resumed. After 47 minutes addition time, the silicate addition was stopped. The acid flow was continued until the batch PH dropped to 7. At pH 7, the acid flow rate was reduced to 2.7 l/min and continued until the batch pH approached 4.6, at which time the acid flow was discontinued. The batch pH was then manually adjusted to 4.6±0.1, and the resultant batch was allowed to digest for 15 minutes at that pH level. The resultant batch was then washed and filtered with EIMCO filter press to 1.5% to 2% sulfate to form a filtered cake which was then press filtered and then re-slurried in a mobile cake slurry tank using the agitator, with minimal amount of water added, to form the initial silica wetcake product.

From this standard wetcake, further samples were then produced.

### INITIAL EXAMPLE 2

ZEODENT® 165 (from J.M. Huber Corporation) was supplied in a feed slurry form. The slurry was then dewatered EIMCO at a fill pressure of 50 psig. The resultant was not further washed, but dried with compressed air for 10 minutes.

From this standard wetcake, further samples were then produced.

### INITIAL EXAMPLE 3

63 liters of 13.3% 2.65 MR sodium silicate were introduced within a reactor and agitated at 40 rpm while heated to 89° C. with a recirculation rate of 30 Hz. At that time, simultaneous addition of sodium silicate (13.3%, 85° C.) and sulfuric acid (11.4%) was initiated with the silicate introduced at a rate of 12.3 l/min and the acid at 5.5 l/min. After 8 minutes of addition time, the agitator was increased to 60 rpm and held there for another 47 minutes, after which the silicate addition was terminated. The acid flow was continued until the pH was about 7.0. At pH 7, the acid flow rate was reduced to 1.5 l/min and continued until the batch pH adjusted to 6.2, at which time the acid flow was discontinued. The batch pH was then manually adjusted to 5.9, and the resultant batch was allowed to digest for 10 minutes at that pH level at 93° C. The resultant batch was then washed and filtered with EIMCO filter press to 1.5% to 2% sulfate to form a filtered cake which was then re-slurried in a mobile cake slurry tank using the agitator, with minimal amount of water added, to form the initial silica wetcake product.

From this standard wetcake, further samples were then produced.

### Wetcake Treatment

### COMPARATIVE EXAMPLE I

The wetcake from Initial Example 1 was mixed thoroughly for 5 minutes. Subsequently, the resultant wetcake was then dried to a target moisture of between 3 and 7%. The dried particles was then milled by a Raymond mill to a particle size of 11.7 μm.

### INVENTIVE EXAMPLE 1

The wetcake from Initial Example 1 was mixed thoroughly for 5 minutes with sodium tetraborate (dry powder) for a targeted 0.5 wt % per dry silica weight. Subsequently, the resultant wetcake was then dried to a target moisture of between 3 and 7%. The dried particles was then milled by a Raymond mill to a particle size of 13.8 μm.

### INVENTIVE EXAMPLE 2

The wetcake from Initial Example 1 was mixed thoroughly for 5 minutes with sodium tetraborate (dry powder) for a targeted 1.0 wt % per dry silica weight. Subsequently, the resultant wetcake was then dried to a target moisture of between 3 and 7%. The dried particles was then milled by a Raymond mill to a particle size of 11.5 μm.

### COMPARATIVE EXAMPLE II

The wetcake from Initial Example 2 was mixed thoroughly for 5 minutes. Subsequently, the resultant wetcake was then dried to a target moisture of between 3 and 7%. The dried particles was then milled by a Raymond mill to a particle size of 10.0 μm.

### INVENTIVE EXAMPLE 3

The wetcake from Initial Example 2 was mixed thoroughly for 5 minutes with sodium tetraborate (dry powder) for a targeted 0.5 wt % per dry silica weight. Subsequently, the resultant wetcake was then dried to a target moisture of between 3 and 7%. The dried particles was then milled by a Raymond mill to a particle size of 10.3 μm.

### INVENTIVE EXAMPLE 4

The wetcake from Initial Example 2 was mixed thoroughly for 5 minutes with sodium tetraborate (dry powder) for a targeted 1.0 wt % per dry silica weight. Subsequently, the resultant wetcake was then dried to a target moisture of between 3 and 7%. The dried particles was then milled by a Raymond mill to a particle size of 10.0 μm.

### COMPARATIVE EXAMPLE III

The wetcake from Initial Example 3 was mixed thoroughly for 5 minutes. Subsequently, the resultant wetcake was then dried to a target moisture of between 3 and 7%. The dried particles was then milled by a Raymond mill to a particle size of 11.4 μm.

### INVENTIVE EXAMPLE 5

The wetcake from Initial Example 3 was mixed thoroughly for 5 minutes with tetrasodium pyrophosphate (dry powder) for a targeted 0.5 wt % per dry silica weight. Subsequently, the resultant wetcake was then dried to a target moisture of between 3 and 7%. The dried particles was then milled by a Raymond mill to a particle size of 12.7 μm.

### INVENTIVE EXAMPLE 6

The wetcake from Initial Example 3 was mixed thoroughly for 5 minutes with tetrasodium pyrophosphate (dry powder) for a targeted 1.0 wt % per dry silica weight. Subsequently, the resultant wetcake was then dried to a target moisture of between 3 and 7%. The dried particles was then milled by a Raymond mill to a particle size of 11 μm.

After being prepared as set forth above, several properties of the particulate silicas and silicates, including % moisture, % 325 residue, % Na₂SO₄, particle size, and oil absorption were then measured. The silica and silicate properties described herein were measured as follows:

Percent moisture was determined by weighing a 2.0 gram sample into a pre-weighed dish to the nearest 0.0001 g. The sample was placed in an oven for 2 hours at 105° C., removed and allowed to cool in a desiccator, then weighed. Percent moisture was determined by dividing the weight loss by the original sample weight and multiplying by 100.

The %325 sieve residue was determined by weighing 50 g silica into a 1-liter beaker containing 500-600 ml water. The silica was allowed to settle into the water, then mixed well until all the material is dispersed. The water pressure was adjusted through the spray nozzle (Fulljet 9.5, ⅜ G, 316 stainless steel, Spraying Systems Co.) to 20-25 psi. The sieve screen (325 mesh screen (45 μm), 8″ diameter) was held 4-6 inches below the nozzle and, while spraying, the contents of the beaker were gradually poured onto the 325 mesh screen. The remaining material was rinsed from the walls of the beaker and poured onto the screen. The screen was washed for 2 minutes, moving the spray from side to side in the screen using a sweeping motion. After spraying for 2 minutes, the residue retained on the screen was washed to one side, and then transferred it into a pre-weighed aluminum weighing dish by washing with water from a squirt bottle. The dish was allowed to stand 2-3 minutes (residue settles), then the clear water off the top is decanted. The dish was placed in an oven (“Easy-Bake” infrared oven or 105° C. oven) and dried until the residue sample reached a constant weight. The dry residue sample and dish were re-weighed. Calculation of the % 325 residue was done as follows:

$$\%325 residue = \frac{weight of residue , g}{sample weight , g} \times 100$$

The 5% pH is determined by weighing 5.0 grams silica into a 250-mL beaker, adding 95 mL deionized or distilled water, mixing for 7 minutes on a magnetic stir plate, and measuring the pH with a pH meter which has been standardized with two buffer solutions bracketing the expected pH range.

Percent sodium sulfate was determined by weighing 13.3 g of silica product or 12.5 g silicate sample and adding it to 240 ml of distilled water. The slurry was mixed for 5 minutes on a Hamilton Beach mixer. The slurry was transferred to a 250-ml graduated cylinder and distilled water is added to make 250 ml slurry. The sample was mixed and the temperature of the slurry is determined. The conductivity of the solution was measured using a Soul-Bridge with the temperature compensator properly adjusted. The percent sulfate was determined from a standard calibration chart.

The Median Particle Size (MPS) was determined using a Horiba LA310 particle size analyzer. A laser beam is projected through a transparent cell which contains a stream of moving particles suspended in a liquid. Light rays that strike the particles are scattered through angles that are inversely proportional to their sizes. The photodetector array measures the quantity of light at several predetermined angles. Electrical signals proportional to the measured light flux values are then processed by a microcomputer system to form a multi-channel histogram of the particle size distribution.

Oil absorption, using linseed oil, was determined by the rubout method. This method is based on a principle of mixing oil with silica by rubbing with a spatula on a smooth surface until a stiff putty-like paste is formed. By measuring the quantity of oil required to have a paste mixture which will curl when spread out, one can calculate the oil absorption value of the silica, which is the value which represents the volume of oil required per unit weight of silica to saturate the silica sorptive capacity. Calculation of the oil absorption value was done as follows:

$$\begin{matrix}Oil absorption = \frac{ml oil absorbed}{weight of silica , in grams} \times 100 \\ = ml oil / 100 gram silica\end{matrix}$$

The Brass Einlehner Abrasion test used to measure the hardness of the precipitated silicas/silica gels reported in this application is described in detail in U.S. Pat. No. 6,616,916, incorporated herein by reference, involves an Einlehner AT-1000 Abrader generally used as follows: (1) a Fourdrinier brass wire screen is weighed and exposed to the action of a 10% aqueous silica suspension for a fixed length of time; (2) the amount of abrasion is then determined as milligrams brass lost from the Fourdrinier wire screen per 100,000 revolutions. The result (listed as Einlehner below), measured in units of mg loss, can be characterized as the 10% brass Einlehner abrasion value.

To measure brightness, fine powder materials pressed into a smooth surfaced pellet were evaluated using a Technidyne Brightmeter S-5/BC. This instrument has a dual beam optical system where the sample is illuminated at an angle of 45°, and the reflected light viewed at 00. It conforms to TAPPI test methods T452 and T646, and ASTM Standard D985. Powdered materials are pressed to about a 1 cm pellet with enough pressure to give a pellet surface that is smooth and without loose particles or gloss.

As a first step in measuring the refractive index (RI) and degree of light transmission, a range of glycerin/water stock solutions (about 10) was prepared so that the refractive index of these solutions lies between about 1.428 and 1.46. The exact glycerin/water ratios needed depend on the exact glycerin used and is determined by the technician making the measurement. Typically, these stock solutions will cover the range of 71 to 91 wt. % glycerin in water. To determine Refractive Index, one or two drops of each standard solution are separately placed on the fixed plate of the refractometer (Abbe 60 Refractometer Model 10450). The covering plate is fixed and locked into place. The light source and refractometer are switched on and the refractive index of each standard solution is read.

Into separate 20 cm³ bottles, 2.0 g+0.01 silica is accurately weighed and 18.0 g+0.01 of each respective stock glycerin/water solution is added to the silica. The bottles are then shaken vigorously to form silica dispersions, the stoppers removed from the bottles, and the bottles placed in a desiccator, which is then evacuated utilizing a vacuum pump. The dispersions are de-aerated for 120 minutes and visually inspected for complete de-aeration. The % transmittance (% T) at 590 nm (Spectronic 20 D+) is measured, after the samples return to room temperature (about 10 minutes) according to the manufacturer's operating instructions. Specifically, % Transmittance is measured on the silica/glycerin/water dispersions by placing an aliquot of each dispersion in a glass spectronic tube and reading the % T at 590 nm wavelength for each sample on a 0-100 scale. % Transmittance vs. RI of the stock solutions used is plotted. Refractive index of the silica is defined as the position of the plotted peak maximum and the value of the peak maximum is the silica % Transmittance.

The results of these measurements and tests for the Inventive Examples are provided below in TABLE IV.

TABLE IV

| | Comparative | Inventive | Inventive |
|---|---|---|---|
| | Example 1 | Example 1 | Example 2 |
| Amt. of Boron | <5 ppm | 518 ppm | 1058 ppm |
| Amt. of Borate | <20 ppm | 1859 ppm | 3799 ppm |
| % Moisture | 3.60% | 3.20% | 2.90% |
| Median Particle Size | 11.7 μm | 13.8 μm | 11.5 μm |
| Na₂SO₄ | 0.35% | 0.35% | 901.00% |
| Oil Absorption | 60 cc/100 g | 58 cc/100 g | 66 cc/100 g |
| pH 5% | 6.64 | 7.25 | 7.34 |
| Technidyne Brightness | 95.0 | 96.4 | 96.8 |
| Einlehner (/100k rev) | 9.856 mg | 9.442 mg | 12.662 mg |
| % Transmittance (glycerin) | 25.6 | 27.8 | 30.9 |
| Refractive Index | 1.445 | 1.441 | 1.445 |
| | Comparative | Inventive | Inventive |
| | Example 2 | Example 3 | Example 4 |
| Amount of Borate | 307 ppm | 2874 ppm | 6275 ppm |
| % Moisture | 10.1% | 3.8% | 6.3% |
| Median Particle Size | 10.0 μm | 10.3 μm | 10.0 μm |
| Na₂SO₄ | 2.87% | 2.32% | 1.45% |
| Oil Absorption | 201 cc/100 g | 213 cc/100 g | 202 cc/100 g |
| pH 5% | 7.01 | 7.09 | 6.94 |
| Technidyne Brightness | 94.8 | 97.6 | 96.4 |
| Einlehner (/100k rev) | 2.289 mg | 1.852 mg | 1.622 mg |
| % Transmittance (glycerin) | 86.2 | 86.2 | 87.2 |
| Refractive Index | 1.448 | 1.448 | 1.445 |

| | Comparative | Inventive | Inventive |
|---|---|---|---|
| | Example 3 | Example 5 | Example 6 |
| Amt. of Phosphate | <0.005% | 0.14% | 0.37% |
| % Moisture | 6.10% | 4.90% | 4.70% |
| Median Particle Size | 11.4 μm | 12.7 μm | 11.0 μm |
| Na₂SO₄ | <0.35% | 0.51% | 0.66% |
| Oil Absorption | 85 cc/100 g | 87 cc/100 g | 87 cc/100 g |
| pH 5% | 7.97 | 7.93 | 7.77 |
| Technidyne Brightness | 94.8 | 94.4 | 91.6 |

The amount of borate exceeded at least 50 ppm (0.005%), and measured as high as 3800 ppm (0.38%) on the silica materials. The amount of phosphate exceeded 0.005% of the weight of the silica materials, and was as high as 0.37% thereof. These borate and phosphate residues remained on the surfaces of the target silicas and were not removed prior to incorporation within a dentifrice.

To demonstrate their efficacy in consumer products, the inventive silicas of Examples 1 and 2 were incorporated as abrasive powders into toothpaste compositions, 1 and 2, respectively), which are set forth in Table V, below.

The performance of toothpaste Compositions 1 and 2 was then compared to each other as were toothpaste Compositions Comparative 1 and Comparative 2. Toothpaste Composition 1 was formulated with Example 1 silica abrasive made according to the inventive process, while Toothpaste Composition Comparative 1 contained Comparative Example 1 abrasive silica made according to a conventional process. Toothpaste Composition 2 was formulated with Example 2 inventive thickener silica, while Toothpaste Composition Comparative 2 contained Comparative Example 2 thickener silica made according to a conventional process.

The toothpaste compositions were prepared as follows. A first mixture was formed by combining at least some of the following components: glycerine, sorbitol, polyethylene glycol (such as CARBOWAX® 600, from the Union Carbide Corporation), polymer thickeners (such CARBOPOL® 940, from Lubrizol Corporation), carboxymethylcellulose (CEKOL® 2000, from CPKelco Oy), xanthan gum (KELDENT®, from CPKelco Oy), and silica thickener (ZEODENT® 165, from J.M. Huber Corporation) and then stirring the first mixture until the components dissolved. A second mixture was formed by combining the following components: deionized water, tetrasodium pyrophosphate, sodium saccharin, sodium fluoride, and then stirring until the components are dissolved. The first and second mixtures were then combined while stirring. Thereafter, color, if specified, was added and the combined mixture is stirred with a Lightnin mixer to obtain a “Pre-mix”.

The premix was placed into a Ross mixer (model 130LDM, Charles Ross & Co., Hauppauge, N.Y.), silica thickener, silica abrasive and any required TiO2 added to the premix, and the premix mixed without vacuum. Then 30 inches of vacuum was drawn and each sample mixed for 15 minutes, and then sodium lauryl sulfate and flavor was added. The resulting mixture was stirred for 5 minutes at a reduced mixing speed. The resulting dentifrice composition was sealed in plastic laminate toothpaste tubes and held under appropriate conditions for later testing. The four different toothpaste compositions were prepared according to the following Formulations, wherein the amounts are gram units:

TABLES V and VI
Toothpaste Formulations

| | Formulation # (% by weight) | | | | | |
|---|---|---|---|---|---|---|
| Component | 1 | 2 | 3 | 4 | 5 | 6 |
| Glycerine, | — | — | — | 3.000 | 3.000 | 3.000 |
| 99.5% | | | | | | |
| Sorbitol, 70.0% | 60.00 | 60.00 | 60.00 | 45.34 | 45.34 | 45.34 |
| Deionized | 12.68 | 12.68 | 12.68 | 12.453 | 12.453 | 12.453 |
| Water | | | | | | |
| CARBOWAX | 4.000 | 4.000 | 4.000 | — | — | — |
| 600 | | | | | | |
| S.D. Alcohol | 1.391 | 1.391 | 1.391 | 2.84 | 2.84 | 2.84 |
| 38B | | | | | | |
| CEKOL 2000 | 0.600 | 0.600 | 0.600 | 0.800 | 0.800 | 0.800 |
| Sodium | 0.334 | 0.334 | 0.334 | 0.540 | 0.540 | 0.540 |
| Saccharin | | | | | | |
| Sodium | 0.243 | 0.243 | 0.243 | 0.440 | 0.440 | 0.440 |
| Fluoride | | | | | | |
| ZEODENT ® | 8.00 | 8.00 | 8.00 | 2.50 | 2.50 | 2.50 |
| 165 | | | | | | |
| Comp. | 10.00 | — | — | 17.20 | — | — |
| Example 1 | | | | | | |
| Inventive Ex. 1 | — | 10.00 | — | — | 17.20 | — |
| Inventive Ex. 2 | — | — | 10.00 | — | — | 17.20 |
| Sodium | — | — | — | 10.00 | 10.00 | 10.00 |
| Bicarbonate | | | | | | |
| Red Color # | 0.300 | 0.300 | 0.300 | — | — | — |
| 33/40 | | | | | | |
| Titanium | — | — | — | 0.300 | 0.300 | 0.300 |
| dioxide | | | | | | |
| sodium lauryl | 1.46 | 1.46 | 1.46 | 2.98 | 2.98 | 2.98 |
| sulfate | | | | | | |
| flavor | 1.000 | 1.000 | 1.000 | 1.000 | 1.000 | 1.000 |
| Total - | 100.0 | 100.0 | 100.0 | 100.0 | 100.0 | 100.0 |

| | Formulation # (% by weight) | | |
|---|---|---|---|
| Component | 7 | 8 | 9 |
| Sorbitol, 70.0% | 60.17 | 60.17 | 60.17 |
| Deionized Water | 11.17 | 11.17 | 11.17 |
| CARBOPOL 940 | 0.300 | 0.300 | 0.300 |
| KELDENT | 0.475 | 0.475 | 0.475 |
| KELDENT | 0.130 | 0.130 | 0.130 |
| Tribasic sodium | 1.450 | 1.450 | 1.450 |
| phosphate | | | |
| Monosodium | 0.590 | 0.590 | 0.590 |
| phosphate | | | |
| Sodium Fluoride | 0.243 | 0.243 | 0.243 |
| Comp. Example 3 | 20.00 | — | — |
| Inventive Ex. 5 | — | 20.00 | — |
| Inventive Ex. 6 | — | — | 20.00 |
| Blue #1 1.00% | 0.050 | 0.050 | 0.050 |
| solution | | | |
| Titanium dioxide | 0.525 | 0.525 | 0.525 |
| Sass | 4.00 | 4.00 | 4.00 |
| Flavor | 0.900 | 0.900 | 0.900 |
| Total - | 100.0 | 100.0 | 100.0 |

Certain toothpaste properties were measured for comparison between the different types of tested silicas. Toothpaste viscosity was measured utilizing a Brookfield Viscometer Model RVT equipped with a Helipath T-F spindle and set to 5 rpm by measuring the viscosity of the toothpaste at 25° C. at three different levels as the spindle descends through the toothpaste test sample and averaging the results. The pH values of the toothpaste mixtures encountered in the present invention were be monitored by conventional pH sensitive electrodes. Aesthetic properties of toothpaste (stand-up, separation) were measured visually. About a one inch ribbon of toothpaste was squeezed from a tube onto a piece of ordinary white notebook paper. After waiting 3-5 minutes, aesthetic property observations were recorded. Stand-up refers to the shape of the toothpaste ribbon and relates to the paste's ability to stay on top of a toothbrush without sinking in-between the bristles. Separation refers to the toothpaste Formulation's integrity. Solid and liquid phases of the toothpaste may separate, usually due to too little binder or thickener. Liquid will be visible around the squeezed ribbon of paste if there is separation.

To determine toothpaste fluoride availability (F/A), a soluble fluoride determination method was used. Toothpaste compositions were stored at a specified temperature for a specified length of time in a laminated tube. Thereafter, 10 grams of the toothpaste composition were placed in a 10 ml beaker and 30 grams of distilled water was added. The mixture was stirred to form an uniformly dispersed toothpaste slurry. The slurry was subsequently centrifuged for 10 minutes at 15,000 rpm or until the supernatant was clear. Then 10 ml of the supernatant and 10 ml of pH 8 buffer (0.2 N EDTA/0.2 N THAM (2-amino-2-hydroxymethyl-1,3-propanediol), previously adjusted to pH=8.0 with NaOH) was pipetted into a plastic vial, a magnetic stir bar added and gentle stirring was initiated. The fluoride ion concentration was determined by direct potentiometry with an Orion fluoride electrode (Model 95-09) utilizing 1000 and 100 ppm F standards to set instrument calibration. Fluoride Availability is basically the percent fluoride determined in the supernatant versus that originally added to the toothpaste, based on the toothpaste abrasive loading level. In such a situation, it is generally accepted that the higher the level the better, particularly since low availability levels would indicate waste of expensive fluoride agents.

Each inventive and comparative toothpaste example formulation exhibited acceptable viscosity measurements and viscosity increases over time, proper standup properties when applied to toothbrush bristles, and effectively no separation during three weeks of storage at 80° F. Thereby, in terms of those such properties, the resultant toothpaste Formulations were essentially the same in quality.

TABLE VII
Sodium Borate Effect on Toothpaste

| | 25% | | | Stand- | | Specific | |
|---|---|---|---|---|---|---|---|
| | pH | Texture | Dispersion | Up | % F/A 80° F. | Gravity | Viscosity |
| Time —Initial | | | | | | | 24 hr |
| Formulation 1 | 6.70 | 9 | 9 | 9 | 101 | 1.331 | 150000 cps |
| Formulation 2 | 6.57 | 9 | 9 | 9 | 101 | 1.324 | 120000 cps |
| Formulation 3 | 6.34 | 9 | 9 | 9 | 98 | 1.327 | 150000 cps |
| Formulation 4 | 8.60 | 8 | 9 | 8 | 95 | 1.395 | 120000 cps |
| Formulation 5 | 8.58 | 8 | 9 | 8 | 98 | 1.384 | 120000 cps |
| Formulation 6 | 8.61 | 8 | 9 | 8 | 102 | 1.388 | 50000 cps |
| Stability 1 Week | | | | | | % F/A 120 F. | |
| Formulation 1 | 9 | 9 | 9 | 9 | 102 | 95 | 160000 cps |
| Formulation 2 | 9 | 9 | 9 | 9 | 100 | 98 | 160000 cps |
| Formulation 3 | 9 | 9 | 9 | 9 | 100 | 94 | 180000 cps |
| Formulation 4 | 7 | 6 | 9 | 9 | 99 | 98 | 120000 cps |
| Formulation 5 | 7 | 6 | 9 | 9 | 100 | 102 | 120000 cps |
| Formulation 6 | 7 | 6 | 9 | 9 | 100 | 100 | 30000 cps |
| Stability 3 Week | | | | | | | |
| Formulation 1 | 9 | 8 | 9 | 8 | 99 | 94 | 310000 cps |
| Formulation 2 | 9 | 8 | 9 | 8 | 100 | 93 | 300000 cps |
| Formulation 3 | 9 | 8 | 9 | 8 | 97 | 92 | 320000 cps |
| Formulation 4 | 8 | 6 | 9 | 9 | 98 | 92 | 220000 cps |
| Formulation 5 | 8 | 6 | 9 | 9 | 97 | 93 | 230000 cps |
| Formulation 6 | 8 | 6 | 9 | 9 | 96 | 92 | 130000 cps |
| Stability 6 Week | | | | | | | |
| Formulation 1 | 9 | 8 | 9 | 9 | — | 91 | 340000 cps |
| Formulation 2 | 9 | 8 | 9 | 7 | — | 89 | 340000 cps |
| Formulation 3 | 9 | 7 | 9 | 9 | — | 88 | 340000 cps |
| Formulation 4 | 7 | 4 | 9 | 9 | — | 90 | 250000 cps |
| Formulation 5 | 8 | 4 | 8 | 9 | — | 92 | 250000 cps |
| Formulation 6 | 8 | 3 | 9 | 9 | — | 90 | 150000 cps |
| Stability 9 Week | | | | | | | |
| Formulation 1 | 9 | 9 | 9 | 9 | 95 | 90 | 320000 cps |
| Formulation 2 | 9 | 8 | 9 | 8 | 95 | 90 | 340000 cps |
| Formulation 3 | 9 | 7 | 9 | 6 | 93 | 88 | 360000 cps |
| Formulation 4 | 8 | 3 | 9 | 7 | 99 | 95 | 270000 cps |
| Formulation 5 | 8 | 3 | 9 | 6 | 99 | 96 | 280000 cps |
| Formulation 6 | 8 | 3 | 9 | 7 | 98 | 92 | 190000 cps |

TABLE VIII
TSPP Effect on Toothpaste

| | 25% | | | Stand- | | Specific | |
|---|---|---|---|---|---|---|---|
| | pH | Texture | Dispersion | Up | % F/A 80° F. | Gravity | Viscosity |
| Initial | | | | | | | 24 hr |
| Formulation 7 | 6.95 | 9 | 9 | 9 | 97 | 1.370 | 170000 cps |
| Formulation 8 | 6.96 | 9 | 9 | 9 | 95 | 1.370 | 190000 cps |
| Formulation 9 | 7.00 | 9 | 9 | 9 | 93 | 1.367 | 180000 cps |
| Stability 1 Week | | | | | | % F/A 120 F. | |
| Formulation 7 | 9 | 9 | 9 | 9 | 99 | 99 | 230000 cps |
| Formulation 8 | 9 | 9 | 9 | 9 | 97 | 97 | 210000 cps |
| Formulation 9 | 9 | 9 | 9 | 9 | 95 | 97 | 210000 cps |
| Stability 3 Week | | | | | | | |
| Formulation 7 | 9 | 9 | 9 | 9 | 102 | 100 | 210000 cps |
| Formulation 8 | 9 | 9 | 9 | 9 | 101 | 102 | 210000 cps |
| Formualtion 9 | 9 | 9 | 9 | 9 | 101 | 101 | 210000 cps |
| Stability 6 Week | | | | | | | |
| Formulation 7 | 9 | 9 | 9 | 9 | 102 | 101 | 200000 cps |
| Formulation 8 | 8 | 9 | 9 | 9 | 102 | 101 | 200000 cps |
| Formulation 9 | 9 | 9 | 9 | 9 | 101 | 100 | 220000 cps |
| Stability 9 Week | | | | | | | |
| Formulation 7 | 9 | 9 | 9 | 9 | 76 | 75 | 240000 cps |
| Formulation 8 | 9 | 9 | 9 | 9 | 76 | 76 | 240000 cps |
| Formulation 9 | 9 | 9 | 9 | 9 | 75 | 76 | 230000 cps |

Thus, there are no adverse effects on toothpaste formulations utilizing these borate- and polyphosphate-treated abrasives.

### Effect of Additives on Washed Cake Slurries

Borate- and pyrophosphate-treated slurries were produced at differing levels of treatment to measure the effect each treatment and amount of additive had on the viscosity of the treated wetcake particles themselves. Such viscosity measurements were taken by Brookfield viscometer. The results are tabulated below:

TABLE IX
Viscosity Measurements of Silica versus Borax Additive Levels

| Borate Level (%) | Viscosity Measurement (cps) |
|---|---|
| 0.1 | 9100 |
| 0.2 | 8120 |
| 0.3 | 6730 |
| 0.4 | 4730 |
| 0.5 | 3230 |
| 1.0 | 1300 |
| 1.5 | 630 |

TABLE X
Viscosity Measurements of Silica versus Tetrasodium
pyrophosphate Additive Levels

| Phosphate Level (%) | Viscosity Measurement (cps) |
|---|---|
| 0.05 | 90100 |
| 0.1 | 28100 |
| 0.2 | 23700 |
| 0.3 | 23300 |
| 0.4 | 5700 |
| 0.5 | 15800 |
| 0.6 | 23900 |
| 0.7 | 22800 |
| 0.8 | 23400 |
| 0.9 | 22100 |
| 1.0 | 21900 |

It is thus evident that upon adding increasing amounts of either type of additive, the particle viscosity measurements trended downward, thus providing an highly effective means of imparting a reduced viscosity to such silica materials.

The silicas treated with the inorganic borate-containing and/or polyphosphate-containing solutions thus exhibited the desired low viscosity levels, low charring potential (and thus excellent color measurements), and dentifrice compatibility levels, all while exhibiting, as well, no compromise in desired abrasivity. Thus, a relatively simple, yet highly effective, manner of treating precipitated silica wetcake materials of high solids-content for facilitating transport and reduced energy use levels has been provided.

It will be understood that various changes in the details, materials, and arrangements of the parts which have been described and illustrated herein in order to explain the nature of this invention may be made by those skilled in the art without departing from the principles and scope of the invention as expressed in the following claims.

What is claimed is:

1. A method for preparing precipitated silica particulate materials, said method comprising the steps of:
a) producing a wetcake of precipitated silica;
b) treating said wetcake of step “a” with from 0.001 to 3% by weight thereof said wetcake of a dispersant compound selected from the group consisting of at least one borate compound, at least one polyphosphate compound, and any mixtures thereof; and
c) mixing said treated wetcake of step “b” into a homogeneous slurry of treated particles of borate-treated and/or polyphosphate-treated precipitated silica.

2. The method of claim 1 further including the steps of:
d) drying said treated particles of step “c” to a solids content of at most 99%;
e) comminuting said dried individual particles; and
f) incorporating either said treated particles of step “c” or said comminuted individual particles of step “e” into a dentifrice composition.

3. A dentifrice formulation comprising at least one particle produced by the method of claim 1.

4. A dentifrice formulation comprising at least one particle produced by the method of claim 2.

5. A precipitated silica particle exhibiting at least 50 ppm of borate residues present on the surface thereof.

6. A dentifrice formulation comprising at least one particle of claim 5.

7. A precipitated silica particle exhibiting at least 0.05% by weight of polyphosphate residues present on the surface thereof.

8. A dentifrice formulation comprising at least one particle of claim 7.
